(19) United States
(12) Patent Application Publication
(10) Pub. No.: US 2025/0253014 A1
(43) Pub. Date: Aug. 7, 2025

(54) MULTI-ARMED BANDIT WITH OPTIMUM EXPLORATION-EXPLOITATION DISTRIBUTION PARAMETER

(71) Applicant: INTERNATIONAL BUSINESS MACHINES CORPORATION, ARMONK, NY (US)

(72) Inventor: Djallel BOUNEFFOUF, Poughkeepsie, NY (US)

(21) Appl. No.: 18/432,577

(22) Filed: Feb. 5, 2024

(51) Int. Cl. G16H 10/20

(52) U.S. Cl. CPC G16H 10/20

(57) ABSTRACT

A method, computer program product, and computer system for triggering actions within a multi-armed bandit process. In a current iteration of an iterative process: a vector c^{V}(t) of values of V features is generated; a distribution parameter αₜ is selected by maximizing a function that depends on αₜ and a measure of a probability of success θ_{α}; a set C^{U}(t) of U features is selected by maximizing a function that depends on c^{V}(t) and αₜ; values c^{U+V}(t) of respective features in C^{U+V}(t) are received; an arm k(t) is selected by maximizing a function that depends on c^{U+V}(t) and αₜ; an electromagnetic signal is sent to a hardware machine directing the hardware machine to perform an action of the selected arm k(t); a reward rₖ₍ₜ₎ resulting from the hardware machine having performed the action is received; and updates are performed for the next iteration.

### BACKGROUND

The present invention relates to a multi-armed bandit process, and more specifically, to a multi-armed bandit process in which an exploration-exploitation distribution parameter is optimized based on observed features.

### SUMMARY

Embodiments of the present invention provide a method, a computer program product, and a computer system, for triggering actions within a multi-armed bandit process in which an exploration-exploitation distribution parameter is optimized based on observed features.

One or more processors of a computer system sequentially perform iterations t (t=0, 1, . . . , T), wherein T≥2.

Performing time step 0 includes: receiving input comprising: C=set of N features (C₁, . . . , C_{N}) wherein N≥3; a set A of N candidate exploration-exploitation distribution parameter values αᵢ (i.e., α₁, . . . α_{N}); C^{V}=set of V features within C; C^{P}=pool of P features within C such that C=C^{V}+C^{P}, N=V+P, V≥1, and P≥2; U=number of features dynamically selected from C^{P} in each iteration t of an iterative process such that U<P where C^{U}(t) is defined as a set of the U features selected at iteration t; λ(t) at iteration t; constant w such that 0≤w≤1; set of K arms such that K≥2.

The following steps are performed in iteration t (t=1, 2, . . . , N).

A vector c^{V}(t) of dimension N is generated from V values received from an external system that is external to the computer system.

Distribution parameter αₜ is selected from (α₁, . . . α_{N}) by having the selected αₜ maximize a function of α that includes a dependence on c^{V}(t) and θ_{α}, wherein θ_{α} is a measure of a probability of success at each α∈A as measured by rewards observed in response to selections of arms in the set of K arms.

A set C^{U}(t) of the U features is selected from C^{P} by having the selected U features maximize a function that depends on c^{V}(t) and αₜ.

Values c^{U+V}(t) of respective features in C^{U+V}(t) are received from the external system, wherein C^{U+V}(t) is a vector of dimension N and includes C^{U}∪C^{V}(t).

An arm k(t) is selected from the K arms, by having the selected arm k(t) maximize a function that depends on c^{U+V}(t) and αₜ.

An electromagnetic signal is sent to a hardware machine, said electromagnetic signal including the selected arm k(t) and directing the hardware machine to perform an action of the selected arm k(t).

An identification of a reward rₖ₍ₜ₎ resulting from the hardware machine having performed the action of the selected arm k(t) is received, wherein 0≤rₖ₍ₜ₎≤1.

Parameters for the next iteration t+1 is updated if t<T, wherein the parameters being updated include parameters being updated in dependence on rₖ₍ₜ₎, αₜ, or both rₖ₍ₜ₎ and αₜ.

### BRIEF DESCRIPTION OF THE DRAWINGS

FIG. 1 is a flow chart of a method for triggering actions in a sequence of iterations within a multi-armed bandit process, in accordance with embodiments of the present invention.

FIGS. 2A and 2B are flow charts of a process for determining an optimum exploration-exploitation distribution parameter, in accordance with embodiments of the present invention.

FIG. 3 is a flow chart describing a process for selecting a set of features, in accordance with embodiments of the present invention.

FIG. 4 is a flow chart describing a process for selecting an arm, in accordance with embodiments of the present invention.

FIG. 5 is a flow chart describing a process for updating parameters in each iteration in FIG. 1, in accordance with embodiments of the present invention.

FIGS. 6A-6E depict multiple embodiments of interaction among a computer system, an external system, and a hardware machine, in accordance with embodiments of the present invention.

FIG. 7 illustrates a computer system, in accordance with embodiments of the present invention.

FIG. 8 depicts a computing environment which contains an example of an environment for the execution of at least some of the computer code involved in performing the inventive methods, in accordance with embodiments of the present invention.

### DETAILED DESCRIPTION

### 1. Introduction

Embodiments of the present invention present a problem of learning parameters of exploration exploitation trade-off in the contextual bandit problem with a linear reward function setting. In the traditional algorithms that solve the contextual bandit problem, the exploration is a parameter that is tuned by the user. Embodiments of the present invention use algorithms that learn to choose the right exploration parameters in an online manner based on the observed context, and an immediate reward received in response to a selected action. Embodiments of the present invention find the optimal exploration of the contextual bandit algorithm, which is a first step toward the automation of the multi-armed bandit algorithm.

In sequential decision problems such as clinical trials or recommender systems, a decision-making algorithm selects among several actions at each given time-point. Each of these actions is associated with side information, or context (e.g., a user's profile), and the reward feedback is limited to the selected action. For example, in clinical trials, the actions correspond the treatment options being compared, the context is the patient's medical record (e.g., health condition, family history, etc.), and the reward represents the outcome (successful or not) of a selected treatment. In this setting in which the treatments are drugs, it is attempted to have a good trade-off between the exploration of the new drug and the exploitation of the known drug.

This inherent exploration-exploitation trade-off exists in many sequential decision problems and is traditionally modeled as a multi-armed bandit (MAB) problem, stated as follows: there are K “arms” (possible actions), each arm associated with a fixed but unknown reward probability distribution. An “arm” is one action of available actions that may be taken as a result of a decision arrived at under uncertainty. At each step, an arm is selected (i.e., an action is selected) and in response, a reward is received. This reward is drawn according to the selected arm's law and is independent of the previous actions.

A particularly useful version of MAB is the contextual multi-armed bandit problem. In each iteration before choosing an arm, a feature vector, or context, associated with each arm is observed. The learner uses these contexts (i.e., features), along with the rewards of the arms played in the past, to choose which arm to play in the current time iteration. Over time, the objective is to collect enough information about the relationship between the context vectors and rewards, so that the next best arm to play can be predicted by looking at the corresponding contexts (feature vectors). One smart solution for the contextual bandit is the linear upper confidence bound (LINUCB) algorithm, which is based on online ridge regression, and takes the concept of upper-confidence bound to strategically balance exploration and exploitation, using a exploration parameter (a) which essentially controls the tradeoff between exploration and exploitation. A higher value of α encourages more exploration, so that LINUCB is more likely to select actions that have not been explored much. A lower value of α prioritizes exploitation.

However, it is difficult to decide in advance the optimal value of α. Embodiments of the present invention present an algorithm, named COmbLINUCB, that finds the right subset of features to select and computes the optimal value of α in both stationery and non-stationary (switching) environments by adaptively balancing exploration and exploitation according to the context.

### 2. Key Notion

The Contextual Bandit Problem is as follows. At each time point (iteration) t∈{1, . . . , T} where T denotes the total number of time points, a player is presented with a context (feature vector) c(t)∈R^{N} before choosing an arm k∈A={1, . . . , K}, where K denotes the total number of arms. C={C₁, . . . , C_{N}} denotes the set of features (variables) defining the context. Let r=(r₁₍ₜ₎, . . . , r_{K(t)}) denote a reward vector, where rₖ(t) is a reward at time t associated with the arm k∈1, . . . , K. Primary focus, in one embodiment, is on the Bernoulli bandit with binary reward; i.e. rₖ(t)∈{0, 1}. Let π: C→A denote a policy. It is assumed that the expected reward is a linear function of the context c(t), i.e. E[rₖ(t)|c(t)]=μₖ^{T} c(t), where μₖ^{T} is an unknown weight vector (to be learned from the data) associated with the arm k.

Thompson Sampling (TS), also known as Bayesian posterior sampling, is a classical approach to the multi-arm bandit problem, where the reward rₖ(t) for choosing an arm k at time t is assumed to follow a distribution Pr(rₜ|\tilde{μ}) with the parameter \tilde{μ}. Given a prior Pr(\tilde{μ}) on these parameters, their posterior distribution is given by the Bayes rule, Pr(\tilde{μ}|rₜ)∝Pr(rₜ|\tilde{μ}) Pr(\tilde{μ}). A particular case of the Thomson Sampling approach assumes a Bernoulli bandit problem, with rewards being 0 or 1, and the parameters following the Beta prior. Thompson Sampling initially assumes arm k to have prior Beta(1,1) on μₖ (the probability of success). At time t, having observed Sₖ(t) successes (reward=1) and Fₖ(t) failures (reward=0), the algorithm updates the distribution on μₖ as Beta(Sₖ(t), Fₖ(t)). The algorithm then generates independent samples θₖ(t) from these posterior distributions of the μₖ, and selects the arm with the largest sample value.

The feature subset selection approach of embodiments of the present invention is built upon the Contextual Combinatorial Bandit (CCB) problem, specified as follows. Each additional feature Cᵢ∈C^{U} is associated with the corresponding random variable rᵢ(t)∈R which indicates the reward obtained when choosing the i^{th} feature at time t. The reward associated with the set of selected features C^{U}(t) is r_{U}|c^{V}(t)∈R, which is the reward of the selected action k(t) knowing the context vector c^{U+V}(t): r_{U}(t)|c^{V}(t)=rₖ₍ₜ₎|c^{U+V}(t), wherein V denotes the number of features initially observed, U denotes the number of additional features to observe, C^{V} denotes a subset of the features V, C^{U} denotes a subset of the features U, and C^{U+V} denotes a subset of the features U+V. It is assumed that r_{U}|c^{V}(t)=f(rᵢ(t)), Cᵢ∈C^{U}(t), for some reward function f(·). The contextual combinatorial bandit setting can be viewed as a game where a context c^{V}(t) is sequentially observed, subsets C^{U}(t)⊂C are selected, and rewards corresponding to the selected subsets are observed. The reward function f(·) used to compute r_{U}(t) is defined as a sum of the outcomes of the features in C^{U}(t); i.e., r^{U}(t)=Σ_{i∈C}_{^{U}}_{₍ₜ₎} rᵢ(t), although nonlinear rewards can also be used. The objective of the CCB algorithm is to maximize the reward over time. Here a stochastic model is considered, where the expectation of rᵢ(t) observed for a feature i is a linear function of the context vector c^{V}(t): rᵢ(t)|c^{V}(t)=c^{V}(t)^{τ}θᵢ+ϵ_{V}, where θᵢ is an unknown weight vector of size V (to be learned from the data) associated with the feature i and where ϵ_{V} is a zero-mean random vector of size V.

### 3. Algorithm

This section describes an algorithm that learns and use the exploration of the contextual bandit algorithm, namely Algorithm 1 (COmbLINUCB) presented in Table 1.

The COmbLINUCB algorithm solves three levels of multi-armed bandit problems. The first level is the combinatorial multi-armed bandit problem applied to find the parameters of the algorithm. The second level finds the right features. The third level problem is a contextual bandit problem that use the parameters found in the first level to find the optimal arm to play. Let nₐᵢ(t) be the number of times the i-th exploration value has been selected so far. Let rₖ(t) be the reward associated with the arm k at time t. The algorithm takes input that includes the candidate values for the distribution parameter α, as well as the initial values of the Beta distribution parameters in TS. At each iteration t, the values of those Beta distribution parameters, S_{αi}(t) and F_{αi}(t), are updated to represent the current total number of successes and failures, respectively, and then sample the “probability of success” parameter θ_{αi} from the corresponding Beta distribution, separately for each distribution parameter ai to estimate μⁱ, which is the mean reward conditioned to the use of the variable i.

Algorithm 1 in Table 1 is a contextual multi-armed bandit algorithm.

TABLE 1
Algorithm 1: Contextual Bandit Algorithm

1: Input: C = set of N features (C₁, ... , C_{N}) wherein N ≥ 3; set A of N candidate
exploration-exploitation distribution parameter values αᵢ (i.e., α₁, ... α_{N}); C^{V} = set of
V features within C; C^{P} = pool of P features within C such that C = C^{V} + C^{P}, N = V + P,
V ≥ 1, and P ≥ 2; U = number of features dynamically selected from C^{P} in each
iteration t of an iterative process such that U < P where C^{U}(t) is defined as a set of
the U features selected at iteration t; λ(t) at iteration t; constant w such that
0 ≤ w ≤ 1; set of K arms (K ≥ 2)
2. Initialize: ∀k ∈ {1, ... , K}: Aₖ = I_{N}, gₖ = 0_{N}, \hat{μ}ₖ = 0_{N}; ∀i ∈ {1, ... , N},
Bᵢ = I_{N}, zᵢ = 0_{N}, \hat{θ}ᵢ = 0_{N}; A_{α} = I_{N}, b_{α}, = 1_{N}; initial values Sαᵢ(0), Fαᵢ(0) (at t = 0) of Beta
distribution parameters Sαᵢ(t), Fαᵢ(t)
3. Foreach t ∈ 1, 2, ... , T do
4: Observe V values for C^{V} and generate set c^{V}(t) of N values from the V values
5: for all αᵢ, do
6: Sample θαᵢ from Beta(Sαᵢ(t − 1), Fαᵢ(t − 1)) distribution
7: Θ_{α} ← A_{α}⁻¹ * b_{α}
8: p_{t,a} ← Θ_{α}^{T} c^{V}(t) + αᵢ [(c^{V}(t))^{T} A_{α}⁻¹ c^{V}(t)]^{1/2}
9: end for
$10 : Select {α}_{t} = arg max ( w {θ}_{α} + ( 1 - w ) {p}_{t , α} )$ $α ∈ A$
11: Foreach i of feature Cᵢ ∈ C^{P} do
12: sample θᵢ from N(\hat{θ}ᵢ, αₜ²Bᵢ⁻¹) distribution
13: End do
$14 : \begin{matrix}Select {C}^{U} ( t ) = arg max {\sum }_{i ∈ \{ i \}} {{c}^{V} ( t )}^{T} {θ}_{i} & {C}_{i}^{U} = U features {C}_{i} in {C}^{P}\end{matrix}$ $\begin{matrix}{C}_{i}^{U} & \{ i \} = indexes i of U features {C}_{i} in {C}^{P}\end{matrix}$
15: C^{U+V}(t) = C^{V} ∪ C^{U}(t)
16: Observe values c^{U+V}(t) of features in C^{U+V}(t)
17: Foreach arm k = 1, ... , K do sample μₖ from N(\hat{μ}ₖ, aₜ²Aₖ⁻¹) End do
$18 : Select arm k ( t ) = arg max {{c}^{U + V} ( t )}^{T} {μ}_{k}$ $k ⊂ \{ 1 , … , K \}$
19: Observe reward rₖ₍ₜ₎
20: Aₖ = Aₖ + c^{U+V}(t) c^{U+V}(t)^{T}, gₖ = gₖ + C^{U+V}(t)rₖ(t), \hat{μ}ₖ = Aₖ⁻¹gₖ
21: Foreach i ∈ C^{U} do
22: Bᵢ = λ(t)Bᵢ + c^{V}(t)c^{V}(t)^{T}, zᵢ = zᵢ + c^{V}(t) rₖ(t), \hat{θ}ᵢ = λ(t) Bᵢ⁻¹zᵢ
23: End do
24: Sαₜ(t) = Sαₜ(t − 1) + rₖ₍ₜ₎
25: Fαₜ(t) = Fαₜ(t − 1) + (1 − rₖ₍ₜ₎)
26: A_{α} = A_{α} + αₜ c^{V}(t) c^{V}(t)^{T}
27: b_{α} = b^{α} + rₖ₍ₜ₎ αₜ c^{V}(t)

28: End For

Algorithm 1 includes pseudocode encompassing lines 1-28 in which: all matrices are N×N matrices, all vectors are N-dimensional vectors, and superscript T denotes “transpose”.

Line 1 identifies input including: C=set of N features (C₁, . . . , C_{N}) wherein N≥3; set A of N candidate exploration-exploitation distribution parameter values αᵢ (i.e., α₁, . . . α_{N}); C^{V}=set of V features within C; C^{P}=pool of P features within C such that C=C^{V}+C^{P}, N=V+P, V≥1, and P≥2; U=number of features dynamically selected from C^{P} in each iteration t of an iterative process such that U<P where C^{U}(t) is defined as a set of the U features selected at iteration t; λ(t) at iteration t; constant w such that 0≤w≤1; set of K arms (K≥2).

C^{V} consists of any specified V elements of the N elements in C, and C^{P} consists of the remaining elements in C.

For example, if N=8, V=5 and P=3, C^{V} consists of C₁, C₂, C₃, C₄ and C₅, and C^{P} consists of C₆, C₇ and C₈.

As another example, if N=8, V=5 and P=3, C^{V} consists of C₂, C₃, C₅, C₆ and C₈, and C^{P} consists of C₁, C₄ and C₇.

In line 2, various variables and parameters are initialized. The specific initial values in line 2 are illustrative and any other applicable initial values may be used. The meaning of notation used in line 2 is as follows. I_{N} denotes an N×N unit matrix. ON denotes a vector of dimension N wherein each element is zero (0), and I_{N} denotes a vector of dimension N wherein each element is one (1).

Lines 3-28 define the outermost loop of T iterations using iteration index t.

In line 4, V values (V₁, . . . , V_{V}) for respective elements of C^{V} are observed (i.e., received) from an external system, after which an N-dimensional vector c^{V}(t) (i.e., c₁(t), . . . , c_{N}(t)) is generated. The V observed values are elements of c^{V}(t) corresponding to respective elements of C^{V} in C, and the remaining elements in c^{V}(t) are zero (0).

For example, if N=8, V=5 and P=3, and C^{V} consists of C₁, C₂, C₃, C₄ and C₅, and C^{P} consists of C₆, C₇ and C₈, then c^{V}(t)=(0, 0, 0, 0, 0, V₁, V₂, V₃).

As another example, if N=8, V=5 and P=3, and C^{V} consists of C₂, C₃, C₅, C₆ and C₈, and C^{P} consists of C₁, C₄ and C₇, then c^{V}(t)=(V₁, 0, 0, V₄, 0, 0, V₇, 0).

Lines 5-9 define a loop of N iterations over αᵢ; i.e., from α₁ to α_{N}. In iteration i (i.e., αᵢ), θαᵢ is sampled from a Beta(Sαᵢ(t−1), Fαᵢ(t−1)) distribution in line 6, Θ_{α} is computed via A_{α}⁻¹*b_{α} in line 7, and p_{t,a} is computed Θ_{α}^{T} c^{V}(t)+αᵢ [(c^{V}(t))^{T} A_{α}⁻¹ c^{V}(t)]^{1/2} in line 8.

In one embodiment, A_{α}=(D_{α}^{T} D_{α}.+I_{N}), wherein D_{α} is a design matrix of dimension m×N at iteration t, whose rows correspond to m training inputs (e.g., m contexts that are observed previously for arm α), and b_{α}∈R^{m} is the corresponding response vector (e.g., the corresponding m click/no-click user feedback).

Sαᵢ(t) and Fαᵢ(t) are a measure of success and failure, respectively, with respect to rewards received as a result of prior arm selections.

For each αᵢ, θαᵢ and p_{t,a} are each a scalar. θαᵢ is a measure of expect success, and p_{t,a} is a measure of an upper bound of success, as derived from rewards received as a result of prior arm selections

In line 10, αₜ is selected as the αᵢ∈A that maximizes (wθαᵢ+(1−w)p_{t,α}), wherein the input constant w is a weight that determines the relative contributions to αₜ of θαᵢ and p_{t,α}.

In lines 11-13, θᵢ is randomly sampled from a multivariate normal probability distribution N(\hat{θ}ᵢ, α²Bᵢ⁻¹) for each context feature Cᵢ∈C^{P}.

In line 14, C^{U}(t) is selected the set Cᵢ^{U} of U features Cᵢ in C^{P} that maximizes Σ_{i∈{i}} c^{V}(t)^{T}θᵢ wherein {i} denotes the set of indexes i of the U features Cᵢ in C^{P}.

For example, if N=8, V=5, P=3 and U=2, C^{V} consists of C₁, C₂, C₃, C₄ and C₅, and C^{P} consists of C₆, C₇ and C₈, then Cᵢ^{U} is (C₆,C₇), (C₆,C₈), or (C₇,C₈). Thus, C^{U}(t) is selected as whichever of (C₆,C₇), (C₆,C₈), and (C₇,C₈) maximizes Σ_{i∈{i}} c^{V}(t)^{T}θᵢ.

In step 15, C^{U+V}(t) determined as C^{V}∪C^{U}(t).

C^{U+V}(t) is an N-dimensional vector, which requires filling in zero (0) for elements outside of C^{V} and C^{U}. Thus in the preceding example (N=8, V=5, P=3, U=2), if C^{U}(t) is (C₆,C₈), then C^{U+V}(t) is (C₁, C₂, C₃, C₄, C₅, C₆, 0, C₈) wherein zero (0) is inserted into C^{U+V}(t) for element C₇ which is outside of C^{V} and C^{U}.

In step 16, values c^{U+V}(t) of features in C^{U+V}(t) are observed (i.e., received) from the external system.

In step 17, μₖ is randomly sample from a multivariate normal probability distribution N(\hat{μ}ₖ, αₜ²Aₖ⁻¹) for each arm k (k=1, . . . , K).

In step 18, arm k(t) is selected from the set of K arms by maximizing c^{U+V}(t)^{T}μₖ.

In step 19, a reward rₖ₍ₜ₎ is observed (i.e., received) from the external system in response to the selection of arm k(t).

In step 20, Aₖ, gₖ, and \hat{μ}ₖ are updated via: Aₖ=Aₖ+c^{U+V}(t)c^{U+V}(t)^{T}, gₖ=gₖ+c^{U+V}(t)rₖ(t), and \hat{μ}ₖ=Aₖ⁻¹gₖ.

In steps 21-23, for each Cᵢ∈C^{U}, Bᵢ, zᵢ, and \hat{θ}ᵢ are updated via: Bᵢ=λ(t)Bᵢ+c^{V}(t)c^{V}(t)^{T}, zᵢ=zᵢ+c^{V}(t) rₖ(t), and \hat{θ}ᵢ=λ(t) Bᵢ⁻¹ zᵢ.

In steps 24-27, Sαₜ(t), Fαᵢ(t), Aαₜ, and bαₜ are updated via: Sαₜ(t)=Sαₜ(t−1)+rₖ₍ₜ₎, Fαₜ(t)=Fαₜ(t−1)+(1−rₖ₍ₜ₎), Aαₜ=Aαₜ+αₜ c^{V}(t) c^{V}(t)^{T}, and bαₜ=bαₜ+rₖ₍ₜ₎ αₜ c^{V}(t).

### 4. Experimentation

Experiments are next presented for evaluating regret for COmbLINUCB-1 and COmbLINUCB-2 algorithms in both a stationary environment and a non-stationary environment.

### 4.1 Stationary Environment

The stationary environment considers the case of two Bernoulli arms, and the dataset is Adult dataset UCI dataset, and Table 2 shows regret in the stationary environment for the methods of LINUCB (maximum, minimum, mean, median), COmbLINUCB-1, and COmbLINUCB-2 for training set sizes of 0, 1000, 5000, and 10000. COmbLINUCB-1 and COmbLINUCB-2 are COmbLINUCB with w=1 and w=0, respectively. With the stationary environment, the reward function of each arm does not change as the number of iterations is varied. Regret is the expected loss from not taking the optimum action, so that the indicated minimum regret corresponds to the best performance of LINUCB.

The reward is a prediction task to determine whether a person makes over 50K a year. Each person is defined by some categorical and continuous information (age, work class, etc). An interval of possible α is [0.01; 1] with a step of 0.01. The mean, median, minimum, and maximum of empirical average cumulative regret of each LINUCB LINUCB with different a are provided in Table 2. The rewards as a function of each arm are stationary. LINUCB was executed with 100 different values of α, and COmbLINUCB-1 and COmbLINUCB-2 were used with a selection of an a from 100 specified values of α. A comparison of empirical average cumulative regret was made with LINUCB, COmbLINUCB-1 and COmbLINUCB-2.

Table 2 shows that: (i) LINUCB has lower minimum regret than the regret of COmbLINUCB-1 and COmbLINUCB-2, with an exception of LINUCB having a higher minimum regret than the regret of COmbLINUCB-2 at a training size of 10000; and (ii) COmbLINUCB-1 and COmbLINUCB-2 have a lower regret than the mean regret and median regret of LINUCB, with an exception of LINUCB having lower mean and median regret than the regret of COmbLINUCB-2 at a training size of 10000.

TABLE 2
Regret in a stationary environment for α ∈ [0.01, 1]

| Training set size | 0 | 1000 | 5000 | 10000 |
|---|---|---|---|---|
| LINUCB maximum regret | 5216 | 5116 | 4406 | 3658 |
| LINUCB minimum regret | 5096 | 4964 | 4297 | 3560 |
| LINUCB mean regret | 5152.1 | 5035.17 | 5035.17 | 3605.63 |
| LINUCB median regret | 5149 | 5031 | 5031 | 3605 |
| COmbLINUCB-1 regret | 5121 | 4981 | 4334 | 3571 |
| COmbLINUCB-2 regret | | 5014 | 4399 | 3654 |

### 4.2 Non-Stationary Environment

The non-stationary environment provides a challenging setting in which the reward function of each arm changes at a fixed number of iterations. The training set has 5000 items. Table 3 shows cumulative regret for a non-stationary reward function of each arm that changes at 100, 1000, and 10000 iterations.

Table 3 shows that: (i) COmbLINUCB-2 has lower regret than the minimum, mean and median regret of LINUCB at 100 and 1000 iterations; (ii) COmbLINUCB-1 has higher regret than the minimum, mean and median regret of LINUCB at 100 and 1000 iterations; and (iii) LINUCB has lower minimum, mean, and median regret than the regret of COmbLINUCB-1 and COmbLINUCB-2 at 100, 1000, and 10000 iterations.

TABLE 3
Performance of regret in a non-stationary environment
for α ∈ [.01, 1]

| Switch iteration | 100 | 1000 | 10000 |
|---|---|---|---|
| LINUCB maximum regret | 16123 | 15970 | 11063 |
| LINUCB minimum regret | 15869 | 15331 | 9291 |
| LINUCB mean regret | 15973.04 | 15584.21 | 10179.37 |
| LINUCB median regret | 15960.5 | 15570 | 10200 |
| COmbLINUCB-1 regret | 16061 | 15630 | 12601 |
| COmbLINUCB-2 regret | 13720 | 13572 | 10706 |

### 5. Inventive Methods

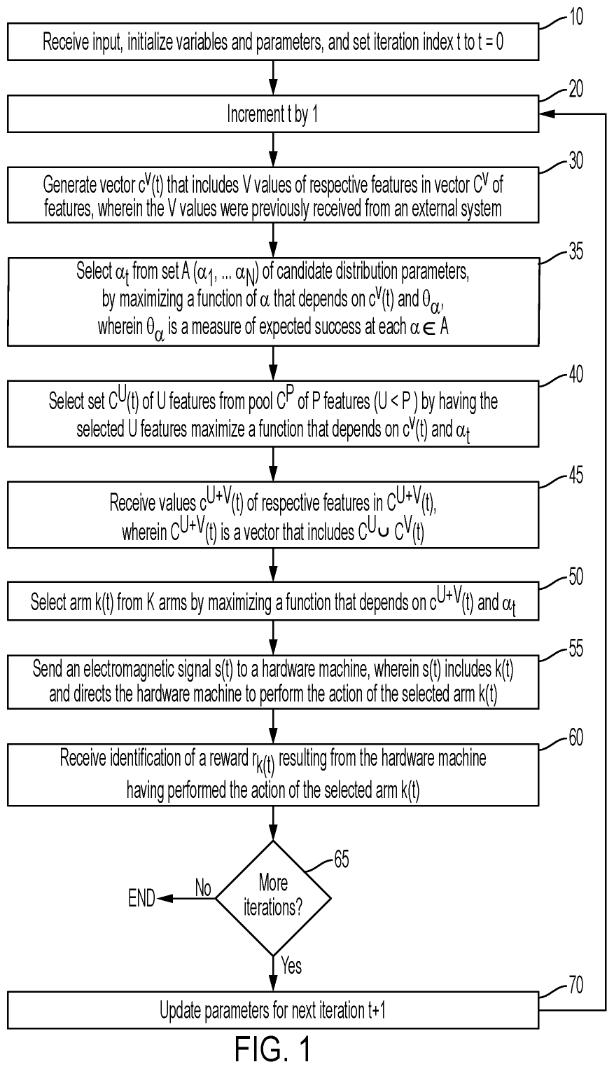

FIGS. 1-8 describe implementations of Algorithm 1 as utilized in embodiments of the present invention. All matrices are N×N matrices, all vectors are N-dimensional vectors, and superscript T denotes “transpose”, where N≥3.

FIG. 1 is a flow chart of a method for triggering actions in a sequence of iterations within a multi-armed bandit process, in accordance with embodiments of the present invention.

The method of FIG. 1 includes steps 10-70 which perform, by one or more processors of a computer system, iterations t (t=0, 1, . . . , T) of an iterative process, wherein T≥2. Thus, the total number of iterations is T+1.

Step 10 receives input, initializes variables and parameters, and sets iteration t to t=0.

The input received in step 10 includes: C=set of N features (C₁, . . . , C_{N}) wherein N≥3; set A of N candidate exploration-exploitation distribution parameter values αᵢ (i.e., α₁, . . . α_{N}); C^{V}=set of V features within C; C^{P}=pool of P features within C such that C=C^{V}+C^{P}, N=V+P, V≥1, and P≥2; U=number of features dynamically selected from C^{P} in each iteration t of an iterative process such that U<P where C^{U}(t) is defined as a set of the U features selected at iteration t; λ(t) at iteration t; constant w such that 0≤w≤1; set of K arms (K≥2).

C^{V} consists of any specified V elements of the N elements in C, and C^{P} consists of the remaining elements in C.

For example, if N=8, V=5 and P=3, C^{V} consists of C₁, C₂, C₃, C₄ and C₅, and C^{P} consists of C₆, C₇ and C₈.

As another example, if N=8, V=5 and P=3, C^{V} consists of C₂, C₃, C₅, C₆ and C₈, and C^{P} consists of C₁, C₄ and C₇.

The variables and parameters initializations (with illustrative initial values) in step 10 include: ∀k∈{1, . . . , K}: Aₖ=I_{N}, gₖ=0_{N}, \hat{μ}ₖ=0_{N}; ∀i∈{1, . . . , N}, Bᵢ=I_{N}, zᵢ=0_{N}, \hat{θ}ᵢ=0_{N}; A_{α}=I_{N}, b_{α}, =1_{N}; initial values Sαᵢ(0), Fαᵢ(0) (at t=0) of Beta distribution parameters Sαᵢ(t), Fαᵢ(t). The preceding specific initial values are illustrative and any other applicable initial values may be used. The meaning of notation used is as follows. I_{N} denotes an N×N unit matrix. 0_{N} denotes a vector of dimension N wherein each element is zero (0), and 1_{N} denotes a vector of dimension N wherein each element is one (1).

Steps 20-70 define a loop of T iterations using iteration index t such that steps 20-70 are performed in iteration t.

Step 20 increments t by 1.

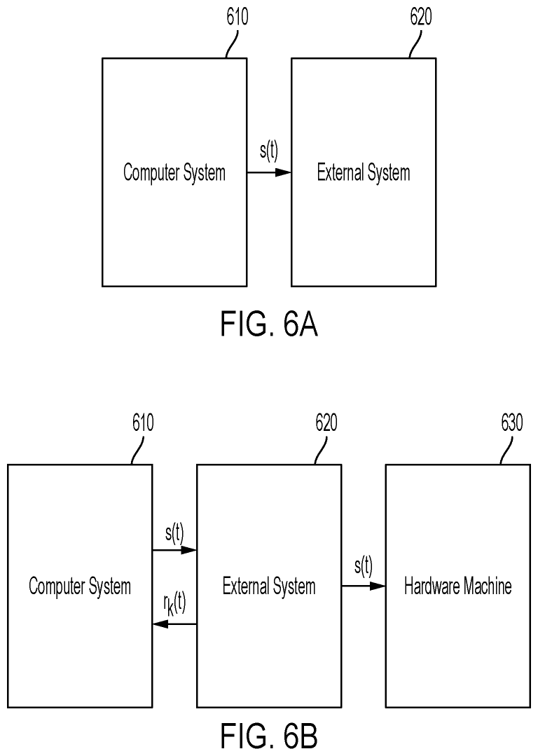

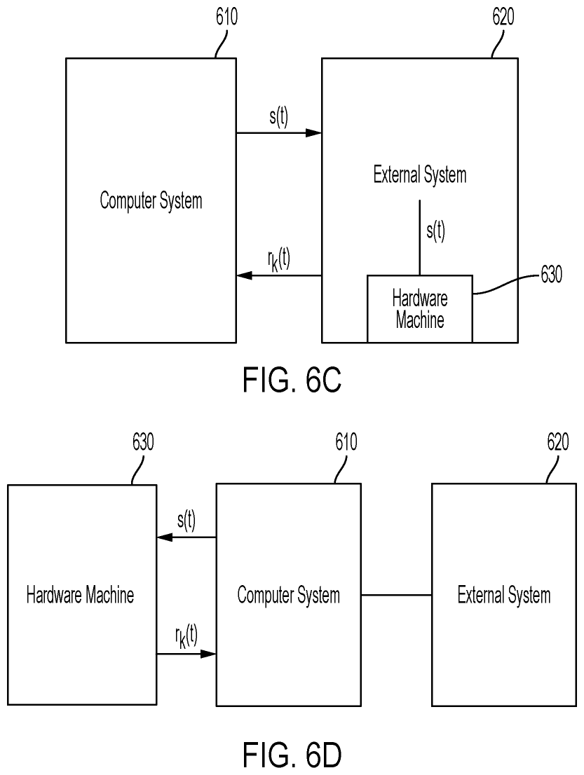

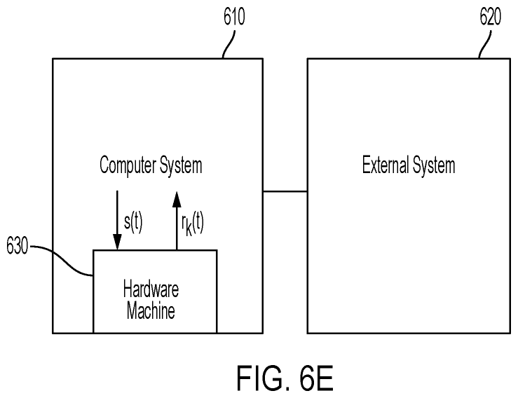

Step 30 generates a vector c^{V}(t) of dimension N from V values (V₁, . . . , V_{V}) received from an external system 620 that is external to a computer system 610 (see FIGS. 6A-6E discussed infra), wherein c^{V}(t) includes the V values of the respective V features in C^{V}. The V observed values are elements of c^{V}(t) corresponding to respective elements of C^{V} in C, and the remaining elements in c^{V}(t) are zero (0).

For example, if N=8, V=5 and P=3, and C^{V} consists of C₁, C₂, C₃, C₄ and C₅, and C^{P} consists of C₆, C₇ and C₈, then c^{V}(t)=(0, 0, 0, 0, 0, V₁, V₂, V₃).

As another example, if N=8, V=5 and P=3, and C^{V} consists of C₂, C₃, C₅, C₆ and C₈, and C^{P} consists of C₁, C₄ and C₇, then c^{V}(t)=(V₁, 0, 0, V₄, 0, 0, V₇, 0).

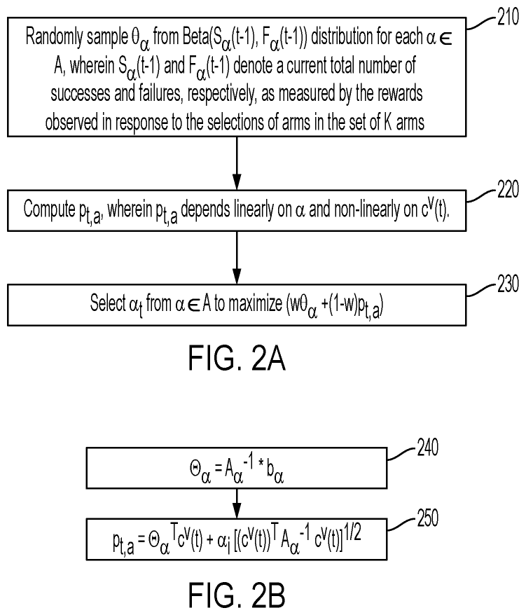

Step 35 selects the optimum exploration-exploitation distribution parameter αₜ from (α₁, . . . α_{N}) by having the selected αₜ maximize a function that depends on c^{V}(t) and θ_{α}, wherein θ_{α} is a measure of expected success at each α∈A as measured by rewards observed in response to selections of arms in the set of K arms. FIG. 2 described infra presents an embodiment for implementing step 35.

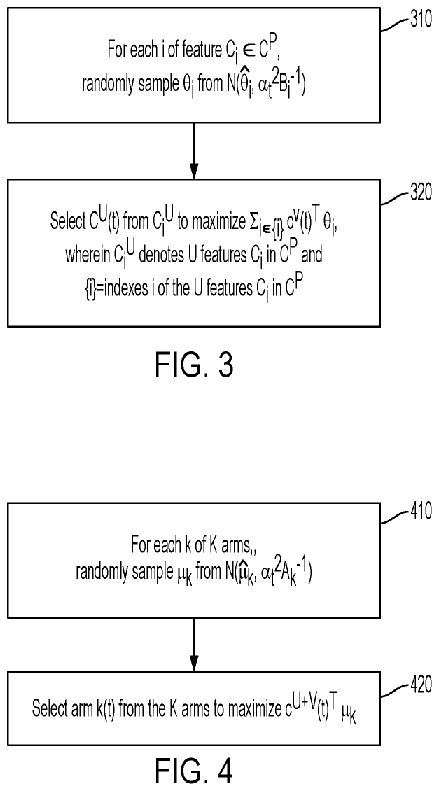

Step 40 selects a set C^{U}(t) of the U features from C^{P} by having the selected U features maximize a function that depends on c^{V}(t) and αₜ. FIG. 3 described infra presents an embodiment for implementing step 40.

Step 45 receives, from the external system 620, values c^{U+V}(t) of respective features in C^{U+V}(t), wherein C^{U+V}(t) is a vector of dimension N and includes C^{U}∪C^{V}(t) and c^{U+V}(t) is a vector of dimension N corresponding to C^{U+V}(t).

Step 50 selects an arm k(t) from the K arms, by having the selected arm k(t) maximize a function that depends on c^{U+V}(t) and αₜ.

Step 55 sends an electromagnetic signal s(t) to a hardware machine. The electromagnetic signal s(t) includes an identification of the selected arm k(t). The electromagnetic signal s(t) directs 630 the hardware machine to perform an action of the selected arm k(t) (see FIGS. 6A-6E described infra).

In one embodiment, the electromagnetic signal s(t) also includes a context c(t) comprising the values c^{U+V}(t) of respective features in C^{U+V}(t) where the values c^{U+V}(t) provide additional information enabling the hardware machine to execute the action in an improved manner that enhances the resultant reward rₖ₍ₜ₎ (see step 60 described infra).

In one embodiment the electromagnetic signal is a wired signal (e.g., via cable).

In one embodiment the electromagnetic signal is a wireless signal via any of, inter alia, Wireless Fidelity (Wi-Fi), Bluetooth technology, Near Field Communication (NFC), Wireless Ethernet, etc.

In one embodiment, the hardware machine 630 is a computer.

In one embodiment, the hardware machine 630 is not a computer.

In one embodiment, the hardware machine 630 is not a generic computer.

In one embodiment, the hardware machine 630 is a specialized machine designed to perform specific functions with high efficiency and accuracy and are optimized for particular tasks, resulting in improved performance and/or reduced power consumption compared to general-purpose machines.

Examples of such specialized machine include, inter alia, an Application-Specific Integrated Circuit (ASIC) which is a custom-designed integrated circuit tailored to perform a specific application or task; Field-Programmable Gate Array (FPGA) which are semiconductor devices that can be programmed and reprogrammed to perform specific tasks after manufacturing; Neural Processing Unit (NPU) which is a specialized hardware accelerator designed to execute neural network models efficiently and may be used, inter alia, artificial (AI) applications; Tensor Processing Unit (TPU) which is a custom-designed AI accelerator optimized for executing machine learning workloads; Graphics Processing Unit (GPU) designed for rendering graphics and may be especially useful in parallel processing tasks due to their ability to handle a large number of calculations simultaneously; Digital Signal Processor (DSP) which is a specialized microprocessor optimized for processing digital signals, such as audio and video.

In one embodiment, the hardware machine 630 performs the action by performing a process selected from the group consisting of a mechanical process, an electrical process, a chemical process, a biological process, and any combination thereof.

Multiple embodiments of interaction among the computer system, the external system, and the hardware machine for implementing steps 55 and 60 are described infra in FIGS. 6A-6E.

Step 60 receives an identification of a reward rₖ₍ₜ₎ resulting from the hardware machine having performed the action of the selected arm k(t), wherein 0≤rₖ₍ₜ₎≤1 (see FIGS. 6A-6E described infra). In one embodiment, the identification of the reward rₖ₍ₜ₎ may be received as an electromagnetic signal.

Step 65 determines whether there are more iterations to be executed. If so (Yes; t<T) then the method performs step 70 followed by looping back to step 20 to perform the next iteration t+1. If not (No; t=T) then the method ends.

Step 70 updates parameters for the next iteration t+1 if t<T. The parameters being updated include parameters being updated in dependence on rₖ₍ₜ₎, αₜ, or both rₖ₍ₜ₎ and αₜ. FIG. 6 described infra presents embodiments for implementing step 70.

FIGS. 2A and 2B are flow charts of a process for determining an optimum exploration-exploitation distribution parameter αₜ, in accordance with embodiments of the present invention. The process of FIGS. 2A and 2B is an embodiment for implementing step 35 of FIG. 1 for selecting αₜ.

The process of FIG. 2A includes steps 210-230.

Step 210 randomly samples θ_{α} from a Beta distribution Beta(S_{α}(t−1), F_{α}(t−1) distribution for each α∈A, wherein S_{α}(t−1) and F_{α}(t−1) denote a current total number of successes and failures, respectively, as measured by the rewards observed in response to the selections of arms in the set of K arms.

Step 220 computes p_{t,α}, wherein p_{t,α} depends linearly on a and non-linearly on c^{V}(t). An embodiment of step 220 is presented in FIG. 2B discussed infra.

Step 230 selects the distribution parameter αₜ from α∈A to maximize (wθ_{α}+(1−w)p_{t,α}), wherein p_{t,α} depends linearly on a and non-linearly on c^{V}(t).

The process of FIG. 2B includes steps 240-250 and is an embodiment of step 220 in FIG. 2A for computing p_{t,α}.

Step 240 computes Θ_{α}=A_{α}⁻¹*b_{α}.

Step 250 computes p_{t,a}=Θ_{α}^{T} c^{V}(t)+αᵢ [((c^{V}(t))^{T} A_{α}⁻¹ c^{V}(t)]^{1/2}, wherein A_{α} is an N×N matrix and b_{α} is vector of order N, and the updating in step 70 of FIG. 1A includes updating A_{α} and b_{α} in each iteration t<T (see FIG. 1 for iterations over t).

FIG. 3 is a flow chart describing a process for selecting a set C^{U}(t) of the U features, in accordance with embodiments of the present invention. The process of FIG. 3 is an embodiment for implementing step 40 of FIG. 1.

The process of FIG. 3 includes steps 310-320.

Step 310 randomly samples θᵢ, for each i of Cᵢ∈C^{P}, from a multivariate normal distribution N(\hat{θ}ᵢ, αₜ²Bᵢ⁻¹), wherein \hat{θ}ᵢ is a vector of order N and Bᵢ is an N×N matrix, and wherein the updating in step 70 of FIG. 1 includes updating \hat{θ}ᵢ and Bᵢ (i=1, . . . , N) in each iteration t<T (see FIG. 1 for iterations over t).

Step 320 selects C^{U}(t) from Cᵢ^{U} to maximize Σ_{i∈{i}} c^{V}(t)^{T}θᵢ, wherein Cᵢ^{U}=U features Cᵢ in C^{P} and {i}=indexes i of the U features Cᵢ in C^{P}.

FIG. 4 is a flow chart describing a process for selecting an arm k(t) from the K arms, in accordance with embodiments of the present invention. The process of FIG. 4 is an embodiment for implementing step 50 of FIG. 1.

The process of FIG. 4 includes steps 410-420.

Step 410 randomly samples μₖ for k=1, . . . , K from a multivariate normal distribution N(\hat{μ}ₖ, αₜ²Aₖ⁻¹), wherein \hat{μ}ₖ is a vector of order N and Aₖ is an N×N matrix, and wherein the updating in step 70 of FIG. 1A includes updating \hat{μ}ₖ and Aₖ in each iteration t<T (see FIG. 1 for iterations over t).

Step 420 selects the arm k(t) from the K arms to maximize c^{U+V}(t)^{T}μₖ.

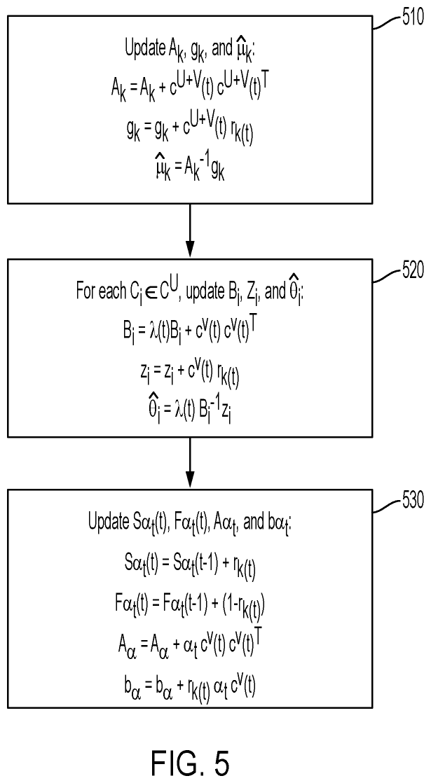

FIG. 5 is a flow chart describing a process for updating parameters in each iteration of the iterative process in FIG. 1, in accordance with embodiments of the present invention. The process of FIG. 5 is an embodiment for implementing step 70 of FIG. 1.

The process of FIG. 5 includes steps 510-530.

Step 510 updates Aₖ, gₖ, and \hat{μ}ₖ as follows: Aₖ=Aₖ+c^{U+V}(t)c^{U+V}(t)^{T}, gₖ=gₖ+c^{U+V}(t)rₖ(t), and \hat{μ}ₖ=Aₖ⁻¹gₖ.

For each Cᵢ∈C^{U}, step 520 updates Bᵢ, zᵢ, and \hat{θ}ᵢ as follows: Bᵢ=λ(t)Bᵢ+c^{V}(t)c^{V}(t)^{T}, zᵢ=zᵢ+c(t) rₖ(t), and \hat{θ}ᵢ=λ(t) Bᵢ⁻¹zᵢ.

Step 530 updates Sαₜ(t), Fαₜ(t), Aαₜ, and bαₜ as follows:

Sαₜ(t)=Sαₜ(t−1)+rₖ₍ₜ₎, Fαₜ(t)=Fαₜ(t−1)+(1−rₖ₍ₜ₎), A_{α}=A_{α} +αₜ c^{V}(t) c^{V}(t)^{T}, and bα=b_{α} +rₖ₍ₜ₎ αₜ c^{V}(t).

FIGS. 6A-6E depict multiple embodiments of interaction among a computer system 610, an external system 620, and a hardware machine 630 for implementing steps 55 and 60 of FIG. 1, in accordance with embodiments of the present invention. The computer system 610 performs embodiments of the method described supra in FIGS. 1-5.

FIG. 6A depicts the computer system 610 sending an electromagnetic signal s(t) to the external system 620 in accordance with step 55 of FIG. 1. The electromagnetic signal identifies the arm k(t) selected in step 50 of FIG. 1 and directs the hardware machine to perform an action of the arm k(t).

FIGS. 6B-6D depict the computer system 610, the external system 620, and the hardware machine 630 in various configuration. In each configuration, the computer system 610 sends the electromagnetic signal s(t) directly or indirectly to the hardware machine 630 in accordance with step 55 of FIG. 1.

In FIG. 6B, the hardware machine 630 is external to both the computer system 610 and the external system 620 and is communicatively coupled to the external system 620. The computer system 610 sends the electromagnetic signal s(t) indirectly to the hardware machine 630, by sending the electromagnetic signal s(t) to the external system 620 followed by the external system 620 subsequently sending the electromagnetic signal s(t) to the hardware machine 630. After the hardware machine 630 performs the action of the arm k(t), the external machine sends to the computer system 610 the reward rₖ(t) or information sufficient for determining the reward rₖ(t) resulting from performance of the action of the arm k(t) by the hardware machine 630.

In FIG. 6C, the hardware machine 630 is internal within the external system 620. The computer system 610 sends the electromagnetic signal s(t) to the hardware machine 630, by (i) sending the electromagnetic signal s(t) directly to the hardware machine 630 or (ii) sending the electromagnetic signal s(t) to a portion of the external system 620 that is external to the hardware machine 630 followed by the external system 620 sending the electromagnetic signal s(t) directly to the hardware machine 630. After the hardware machine 630 performs the action of the arm k(t), the external system 620 sends to the computer system 610 the reward rₖ(t), or information sufficient for determining the reward rₖ(t), resulting from performance of the action of the arm k(t) by the hardware machine 630.

In FIG. 6D, the hardware machine 630 is external to both the computer system 610 and the external system 620 and is communicatively coupled to the computer system 610. The computer system 610 sends the electromagnetic signal s(t) directly to the hardware machine 630. After the hardware machine 630 performs the action of the arm k(t), the hardware machine 630 sends to the computer system 610 the reward rₖ(t) or information sufficient for determining the reward rₖ(t), resulting from performance of the action of the arm k(t) by the hardware machine 630.

In FIG. 6E, the hardware machine 630 is internal within the computer system 610. The computer system 610 sends the electromagnetic signal s(t) to the hardware machine 630. After the hardware machine 630 performs the action of the arm k(t), the hardware machine communicates to the computer system 610 the reward rₖ(t), or information sufficient for determining the reward rₖ(t) resulting from performance of the action of the arm k(t) by the hardware machine 630.

Tables 4-7 are Examples 1-4, respectively, which describe practical applications of embodiments of the present invention.

As stated supra in conjunction with step 55 of FIG. 1, in one embodiment, the electromagnetic signal s(t) also includes a context c(t) comprising the values c^{U+V}(t) of respective features in C^{U+V}(t) where the values c^{U+V}(t) provide additional information enabling the hardware machine to execute the action in an improved manner that enhances the resultant reward rₖ₍ₜ₎. I this embodiment, the reward rₖ₍ₜ₎ reflects the context c(t) included in the electromagnetic signal s(t) sent to the hardware machine. The context c(t) is the “Context” row in Tables 4-7 and is relevant only for embodiments in which the electromagnetic signal includes the context c(t) in addition to the selected arm k(t). For embodiments in which the electromagnetic signal does not include the context c(t), the “Context” row in Tables 4-7 has no effect on the reward rₖ₍ₜ₎. identified in the “Rewards” row in Tables 4-7.

TABLE 4
Example 1

| | |
|---|---|
| Function of Example | routing of packets between nodes of a network or between nodes of |
| | different networks |
| Hardware Machine | network switch (for routing packets within a network) or router (for |
| | routing packets between networks) |
| Context | network traffic volume, network topology, network interference or |
| | noise from other devices |
| Arms/Actions | for a given source node and destination node, selection of which |
| | network path to use for routing the packet from the source node to |
| | the destination node |
| Rewards | network latency (time for a packet to be routed between nodes in a |
| | network or between networks) |

TABLE 5
Example 2

| | |
|---|---|
| Function of Example | selection of printer (from multiple printers in a computer system) to |
| | print the output of a job that was executed by a hardware server in |
| | the computer system |
| Hardware Machine | printer |
| Context | size of the output, print jobs in buffer of each of the multiple printers |
| Arms/Actions | the multiple printers which may print the output |
| Rewards | time from sending the output to the printer to completion of the |
| | printing of the output |

TABLE 6
Example 3

| | |
|---|---|
| Function of Example | control of self-navigating ship sailing in ocean using an on-board |
| | computer to navigate the ship |
| Hardware Machine | self-navigating ship |
| Context | conditions at current location of ship (e.g., ocean waves and |
| | roughness, presence of nearby ships, current ocean depth); |
| | weather conditions (e.g., precipitation, wind speed and direction) |
| Arms/Actions | changing direction of ship motion, accelerating or decelerating |
| | ship motion, invoking ship stabilization apparatus in ship |
| Rewards | optimizing fuel efficiency, minimizing travel time to reach |
| | destination of ship |

TABLE 7
Example 4

| | |
|---|---|
| Function of Example | operation by robotic arms to perform real-time surgery on tissue of a |
| | patient |
| Hardware Machine | robotic arms |
| Context | characteristics of the tissue being treated (bleeding, color, swelling), |
| | patient data (blood pressure, pulse rate, oxygen level, bleeding); |
| | environmental factors (lighting, temperature, humidity) |
| Arms/Actions | change robotic arm operational parameters (motion speed and |
| | direction, power) in each time step |
| Rewards | minimize removal of healthy tissue, minimize duration of each time |
| | step and time of overall process |

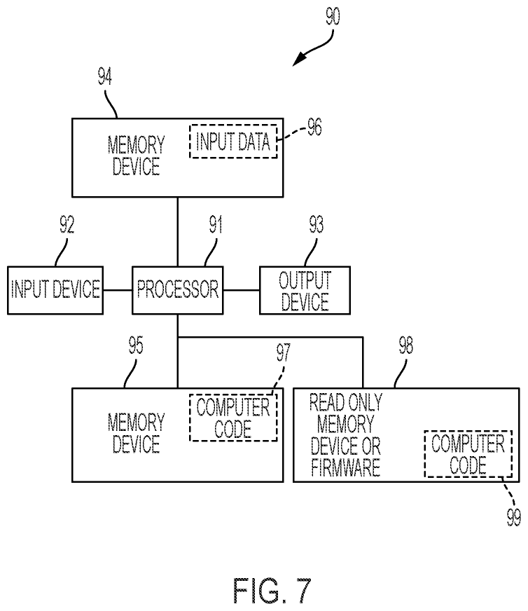

FIG. 7 illustrates a computer system 90, in accordance with embodiments of the present invention.

The computer system 90 includes a processor 91, an input device 92 coupled to the processor 91, an output device 93 coupled to the processor 91, and memory devices 94 and 95 each coupled to the processor 91. The processor 91 represents one or more processors and may denote a single processor or a plurality of processors. The input device 92 may be, inter alia, a keyboard, a mouse, a camera, a touchscreen, etc., or a combination thereof. The output device 93 may be, inter alia, a printer, a plotter, a computer screen, a magnetic tape, a removable hard disk, a floppy disk, etc., or a combination thereof. The memory devices 94 and 95 may each be, inter alia, a hard disk, a floppy disk, a magnetic tape, an optical storage such as a compact disc (CD) or a digital video disc (DVD), a dynamic random access memory (DRAM), a read-only memory (ROM), etc., or a combination thereof. The memory device 95 includes a computer code 97. The computer code 97 includes algorithms for executing embodiments of the present invention. The processor 91 executes the computer code 97. The memory device 94 includes input data 96. The input data 96 includes input required by the computer code 97. The output device 93 displays output from the computer code 97. Either or both memory devices 94 and 95 (or one or more additional memory devices such as read only memory device 96) may include algorithms and may be used as a computer usable medium (or a computer readable medium or a program storage device) having a computer readable program code embodied therein and/or having other data stored therein, wherein the computer readable program code includes the computer code 97. Generally, a computer program product (or, alternatively, an article of manufacture) of the computer system 90 may include the computer usable medium (or the program storage device).

In some embodiments, rather than being stored and accessed from a hard drive, optical disc or other writeable, rewriteable, or removable hardware memory device 95, stored computer program code 99 (e.g., including algorithms) may be stored on a static, nonremovable, read-only storage medium such as a Read-Only Memory (ROM) device 98, or may be accessed by processor 91 directly from such a static, nonremovable, read-only medium 98. Similarly, in some embodiments, stored computer program code 99 may be stored as computer-readable firmware, or may be accessed by processor 91 directly from such firmware, rather than from a more dynamic or removable hardware data-storage device 95, such as a hard drive or optical disc.

Still yet, any of the components of the present invention could be created, integrated, hosted, maintained, deployed, managed, serviced, etc. by a service supplier who offers to improve software technology associated with cross-referencing metrics associated with plug-in components, generating software code modules, and enabling operational functionality of target cloud components. Thus, the present invention discloses a process for deploying, creating, integrating, hosting, maintaining, and/or integrating computing infrastructure, including integrating computer-readable code into the computer system 90, wherein the code in combination with the computer system 90 is capable of performing a method for enabling a process for improving software technology associated with cross-referencing metrics associated with plug-in components, generating software code modules, and enabling operational functionality of target cloud components. In another embodiment, the invention provides a business method that performs the process steps of the invention on a subscription, advertising, and/or fee basis. That is, a service supplier, such as a Solution Integrator, could offer to enable a process for improving software technology associated with cross-referencing metrics associated with plug-in components, generating software code modules, and enabling operational functionality of target cloud components. In this case, the service supplier can create, maintain, support, etc. a computer infrastructure that performs the process steps of the invention for one or more customers. In return, the service supplier can receive payment from the customer(s) under a subscription and/or fee agreement and/or the service supplier can receive payment from the sale of advertising content to one or more third parties.

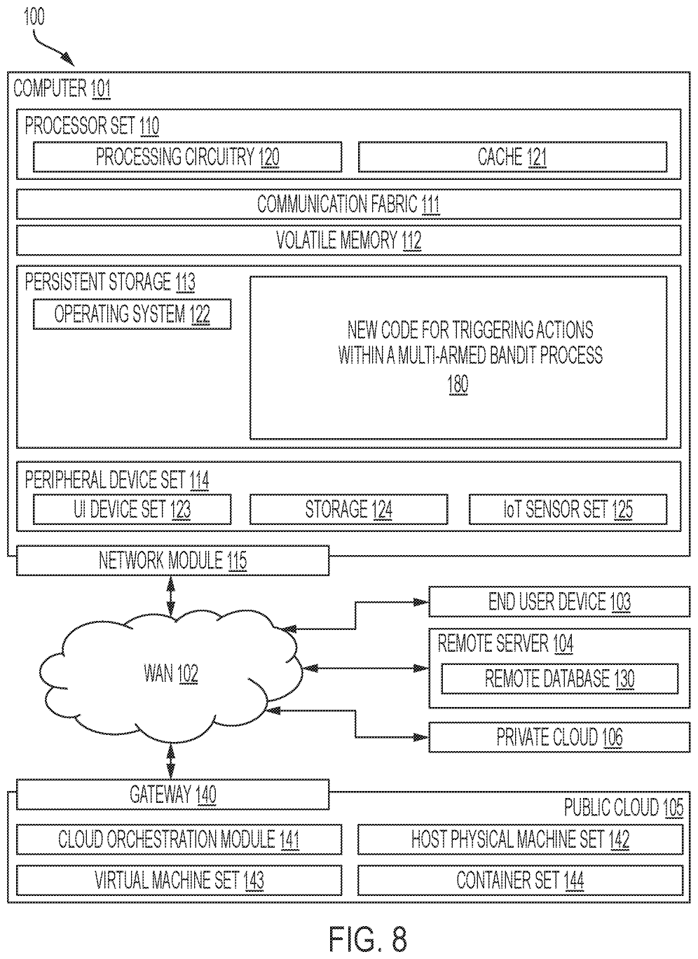

While FIG. 7 shows the computer system 90 as a particular configuration of hardware and software, any configuration of hardware and software, as would be known to a person of ordinary skill in the art, may be utilized for the purposes stated supra in conjunction with the particular computer system 90 of FIG. 8. For example, the memory devices 94 and 95 may be portions of a single memory device rather than separate memory devices.

A computer program product of the present invention comprises one or more computer readable hardware storage devices having computer readable program code stored therein, said program code containing instructions executable by one or more processors of a computer system to implement the methods of the present invention.

A computer system of the present invention comprises one or more processors, one or more memories, and one or more computer readable hardware storage devices, said one or more hardware storage devices containing program code executable by the one or more processors via the one or more memories to implement the methods of the present invention.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer-readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Hash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer-readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

FIG. 8 depicts a computing environment 100 which contains an example of an environment for the execution of at least some of the computer code involved in performing the inventive methods, in accordance with embodiments of the present invention. Such computer code includes new code for triggering actions within a multi-armed bandit process 180. In addition to block 180, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer-readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer-readable program instructions are stored in various types of computer-readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer-readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

CLOUD COMPUTING SERVICES AND/OR MICROSERVICES (not separately shown in FIG. 1): private and public clouds 106 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider's systems, and back. In some embodiments, cloud services may be configured and orchestrated according to as “as a service” technology paradigm where something is being presented to an internal or external customer in the form of a cloud computing service. As-a-Service offerings typically provide endpoints with which various customers interface. These endpoints are typically based on a set of APIs. One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with these things. Another category is Software as a Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for triggering actions within a multi-armed bandit process in which an exploration-exploitation distribution parameter is optimized based on observed features, said method comprising:
sequentially performing, by one or more processors of a computer system, iterations t (t=0, 1, . . . , T), wherein T≥2,
wherein performing iteration 0 includes receiving input comprising: C=set of N features (C₁, . . . , C_{N}) wherein N≥3; a set A of N candidate exploration-exploitation distribution parameter values αᵢ (i.e., α₁, . . . α_{N}); C^{V}=set of V features within C; C^{P}=pool of P features within C such that C=C^{V}+C^{P}, N=V+P, V≥1, and P≥2; U=number of features dynamically selected from C^{P} in each iteration t of an iterative process such that U<P where C^{U}(t) is defined as a set of the U features selected at iteration t; λ(t) at iteration t; constant w such that 0≤w≤1; set of K arms such that K≥2;
wherein performing iteration t (t=1, . . . , T) comprises:
generating a vector c^{V}(t) of dimension N from V values received from an external system that is external to the computer system, wherein c^{V}(t) includes the V values of the respective V features in C^{V};
selecting distribution parameter αₜ from (α₁, . . . α_{N}) by having the selected αₜ maximize a function of α that includes a dependence on c^{V}(t) and θ_{α}, wherein θ_{α} is a measure of a probability of success at each α∈A as measured by rewards observed in response to selections of arms in the set of K arms;
selecting a set C^{U}(t) of the U features from C^{P} by having the selected U features maximize a function that depends on c^{V}(t) and αₜ;
receiving, from the external system, values c^{U+V}(t) of respective features in C^{U+V}(t), wherein C^{U+V}(t) is a vector of dimension N and includes C^{U}∪C^{V}(t);
selecting an arm k(t) from the K arms, by having the selected arm k(t) maximize a function that depends on c^{U+V}(t) and αₜ;
sending an electromagnetic signal to a hardware machine, said electromagnetic signal including the selected arm k(t) and directing the hardware machine to perform an action of the selected arm k(t);
receiving an identification of a reward rₖ₍ₜ₎ resulting from the hardware machine having performed the action of the selected arm k(t), wherein 0≤rₖ₍ₜ₎≤1; and
updating parameters for the next iteration t+1 if t<T, said parameters being updated including parameters being updated in dependence on rₖ₍ₜ₎, αₜ, or both rₖ₍ₜ₎ and αₜ.

2. The method of claim 1, wherein the method comprises in iteration t:
randomly sampling θ_{α} from a Beta distribution Beta(S_{α}(t−1), F_{α}(t−1) for each α∈A, wherein S_{α}(t−1) and F_{α}(t−1) denote a current total number of successes and failures, respectively, as measured by the rewards observed in response to the selections of arms in the set of K arms;
computing p_{t,α}, wherein p_{t,α} depends linearly on a and non-linearly on c^{V}(t).
selecting αₜ from α∈A to maximize (wθ_{α}+(1−w)p_{t,α}).

3. The method of claim 2, wherein said computing p_{t,α} comprises:
computing Θ_{α}=A_{α}⁻¹*b_{α}; and
computing p_{t,a}=Θ_{α}^{T} c^{V}(t)+αᵢ [((c^{V}(t))^{T} A_{α}⁻¹ c^{V}(t)]^{1/2}, wherein Aₐ is an N×N matrix and b_{α} is vector of order N, and wherein said updating comprises updating A_{α} and b_{α} in each iteration t<T.

4. The method of claim 1, wherein the method comprises:
randomly sampling θᵢ, for each i of Cᵢ∈C^{P}, from a normal distribution N(\hat{θ}ᵢ, αₜ²Bᵢ⁻¹) wherein \hat{θ}ᵢ is a vector of order N and Bᵢ is an N×N matrix, and wherein said updating comprises updating \hat{θ}ᵢ and Bᵢ (i=1, . . . , N) in each iteration t<T; and
selecting C^{U}(t) from Cᵢ^{U} to maximize Σ_{i∈{i}} c^{V}(t)^{T}θᵢ, wherein Cᵢ^{U}=U features Cᵢ in C^{P} and {i}=indexes i of the U features Cᵢ in C^{P}.

5. The method of claim 1, wherein the method comprises:
randomly sample μₖ for k=1, . . . , K from a normal distribution N(\hat{μ}ₖ, αₜ²Aₖ⁻¹), wherein \hat{μ}ₖ is a vector of order N and Aₖ is an N×N matrix, and wherein said updating comprises updating \hat{μ}ₖ and Aₖ in each iteration t<T; and
selecting the arm k(t) from the K arms to maximize c^{U+V}(t)^{T}μₖ.

6. The method of claim 1, wherein the hardware machine is not a generic computer.

7. The method of claim 1, wherein the hardware machine is a computing device.

8. The method of claim 1, wherein the hardware machine is an Application-Specific Integrated Circuit (ASIC), a Field-Programmable Gate Array (FPGA), a Neural Processing Unit (NPU), a Tensor Processing Unit (TPU), Graphics Processing Unit (GPU), or Digital Signal Processor (DSP).

9. The method of claim 1, wherein the external system comprises the hardware machine.

10. The method of claim 9, wherein said sending the signal comprises transmitting the electromagnetic signal indirectly to the hardware machine in the external system via a computing device in the external system, said computing device configured to receive the transmitted electromagnetic signal and to subsequently send the transmitted electromagnetic signal to the hardware machine.

11. A computer program product, comprising one or more computer readable hardware storage devices having computer readable program code stored therein, said program code containing instructions executable by one or more processors of a computer system to implement a method for triggering actions within a multi-armed bandit process in which an exploration-exploitation distribution parameter is optimized based on observed features, said method comprising:
sequentially performing, by the one or more processors, iterations t (t=0, 1, . . . , T), wherein T≥2,
wherein performing iteration 0 includes receiving input comprising: C=set of N features (C₁, . . . , C_{N}) wherein N≥3; a set A of N candidate exploration-exploitation distribution parameter values αᵢ (i.e., α₁, . . . α_{N}); C^{V}=set of V features within C; C^{P}=pool of P features within C such that C=C^{V}+C^{P}, N=V+P, V≥1, and P≥2; U=number of features dynamically selected from C^{P} in each iteration t of an iterative process such that U<P where C^{U}(t) is defined as a set of the U features selected at iteration t; λ(t) at iteration t; constant w such that 0≤w≤1; set of K arms such that K≥2;
wherein performing iteration t (t=1, . . . , T) comprises:
generating a vector c^{V}(t) of dimension N from V values received from an external system that is external to the computer system, wherein c^{V}(t) includes the V values of the respective V features in C^{V};
selecting distribution parameter αₜ from (α₁, . . . α_{N}) by having the selected αₜ maximize a function of α that includes a dependence on c^{V}(t) and θ_{α}, wherein θ_{α} is a measure of a probability of success at each α∈A as measured by rewards observed in response to selections of arms in the set of K arms;
selecting a set C^{U}(t) of the U features from C^{P} by having the selected U features maximize a function that depends on c^{V}(t) and αₜ;
receiving, from the external system, values c^{U+V}(t) of respective features in C^{U+V}(t), wherein C^{U+V}(t) is a vector of dimension N and includes C^{U}∪C^{V}(t);
selecting an arm k(t) from the K arms, by having the selected arm k(t) maximize a function that depends on c^{U+V}(t) and αₜ;
sending an electromagnetic signal to a hardware machine, said electromagnetic signal including the selected arm k(t) and directing the hardware machine to perform an action of the selected arm k(t);
receiving an identification of a reward rₖ₍ₜ₎ resulting from the hardware machine having performed the action of the selected arm k(t), wherein 0≤rₖ₍ₜ₎≤1; and
updating parameters for the next iteration t+1 if t<T, said parameters being updated including parameters being updated in dependence on rₖ₍ₜ₎, αₜ, or both rₖ₍ₜ₎ and αₜ.

12. The computer program product of claim 11, wherein the method comprises in iteration t:
randomly sampling θ_{α} from a Beta distribution Beta(S_{α}(t−1), F_{α}(t−1) for each α∈A, wherein S_{α}(t−1) and F_{α}(t−1) denote a current total number of successes and failures, respectively, as measured by the rewards observed in response to the selections of arms in the set of K arms;
computing p_{t,α}, wherein p_{t,α} depends linearly on a and non-linearly on c^{V}(t).
selecting αₜ from α∈A to maximize (wθ_{α}+(1−w)p_{t,α}).

13. The computer program product of claim 12, wherein said computing p_{t,α} comprises in iteration t:
computing Θ_{α}=A_{α}⁻¹*b_{α}; and
computing p_{t,a}=Θ_{α}^{T} c^{V}(t)+αᵢ [((c^{V}(t))^{T} A_{α}⁻¹ c^{V}(t)]^{1/2}, wherein A_{α} is an N×N matrix and b_{α} is vector of order N, and wherein said updating comprises updating A_{α} and b_{α} in each iteration t<T.

14. The computer program product of claim 11, wherein the method comprises in iteration t:
randomly sampling θᵢ, for each i of Cᵢ∈C^{P}, from a normal distribution N(\hat{θ}ᵢ, αₜ²Bᵢ⁻¹) wherein \hat{θ}ᵢ is a vector of order N and Bᵢ is an N×N matrix, and wherein said updating comprises updating \hat{θ}ᵢ and Bᵢ (i=1, . . . , N) in each iteration t<T; and
selecting C^{U}(t) from Cᵢ^{U} to maximize Σ_{i∈{i}} c^{V}(t)^{T}θᵢ, wherein Cᵢ^{U}=U features Cᵢ in C^{P} and {i}=indexes i of the U features Cᵢ in C^{P}.

15. The computer program product of claim 11, wherein the method comprises in iteration t:
randomly sample μₖ for k=1, . . . , K from a normal distribution N(\hat{μ}ₖ, αₜ²Aₖ⁻¹), wherein \hat{μ}ₖ is a vector of order N and Aₖ is an N×N matrix, and wherein said updating comprises updating \hat{μ}ₖ and Aₖ in each iteration t<T; and
selecting the arm k(t) from the K arms to maximize c^{U+V}(t)^{T}μₖ.

16. A computer system, comprising one or more processors, one or more memories, and one or more computer readable hardware storage devices, said one or more hardware storage devices containing program code executable by the one or more processors via the one or more memories to implement a method for triggering actions within a multi-armed bandit process in which an exploration-exploitation distribution parameter is optimized based on observed features, said method comprising:
sequentially performing, by the one or more processors, iterations t (t=0, 1, . . . , T), wherein T≥2,
wherein performing iteration 0 includes receiving input comprising: C=set of N features (C₁, . . . , C_{N}) wherein N≥3; a set A of N candidate exploration-exploitation distribution parameter values αᵢ (i.e., α₁, . . . α_{N}); C^{V}=set of V features within C; C^{P}=pool of P features within C such that C=C^{V}+C^{P}, N=V+P, V≥1, and P≥2; U=number of features dynamically selected from C^{P} in each iteration t of an iterative process such that U<P where C^{U}(t) is defined as a set of the U features selected at iteration t; λ(t) at iteration t; constant w such that 0≤w≤1; set of K arms such that K≥2;
wherein performing iteration t (t=1, . . . , T) comprises:
generating a vector c^{V}(t) of dimension N from V values received from an external system that is external to the computer system, wherein c^{V}(t) includes the V values of the respective V features in C^{V};
selecting distribution parameter αₜ from (α₁, . . . α_{N}) by having the selected αₜ maximize a function of α that includes a dependence on c^{V}(t) and θ_{α}, wherein θ_{α} is a measure of a probability of success at each α∈A as measured by rewards observed in response to selections of arms in the set of K arms;
selecting a set C^{U}(t) of the U features from C^{P} by having the selected U features maximize a function that depends on c^{V}(t) and αₜ;
receiving, from the external system, values c^{U+V}(t) of respective features in C^{U+V}(t), wherein C^{U+V}(t) is a vector of dimension N and includes C^{U}∪C^{V}(t);
selecting an arm k(t) from the K arms, by having the selected arm k(t) maximize a function that depends on c^{U+V}(t) and αₜ;
sending an electromagnetic signal to a hardware machine, said electromagnetic signal including the selected arm k(t) and directing the hardware machine to perform an action of the selected arm k(t);
receiving an identification of a reward rₖ₍ₜ₎ resulting from the hardware machine having performed the action of the selected arm k(t), wherein 0≤rₖ₍ₜ₎≤1; and
updating parameters for the next iteration t+1 if t<T, said parameters being updated including parameters being updated in dependence on rₖ₍ₜ₎, αₜ, or both rₖ₍ₜ₎ and αₜ.

17. The computer system of claim 16, wherein the method comprises in iteration t:
randomly sampling θ_{α} from a Beta distribution Beta(S_{α}(t−1), F_{α}(t−1) for each α∈A, wherein S_{α}(t−1) and F_{α}(t−1) denote a current total number of successes and failures, respectively, as measured by the rewards observed in response to the selections of arms in the set of K arms;
computing p_{t,α}, wherein p_{t,α} depends linearly on a and non-linearly on c^{V}(t).
selecting αₜ from α∈A to maximize (wθ_{α}+(1−w)p_{t,α}).

18. The computer system of claim 17, wherein said computing p_{t,α} comprises in iteration t:
computing Θ_{α}=A_{α}⁻¹*b_{α}; and
computing p_{t,a}=Θ_{α}^{T} c^{V}(t)+αᵢ [((c^{V}(t))^{T} A_{α}⁻¹ c^{V}(t)]^{1/2}, wherein Aₐ is an N×N matrix and b_{α} is vector of order N, and wherein said updating comprises updating A_{α} and b_{α} in each iteration t<T.

19. The computer system of claim 16, wherein the method comprises in iteration t:
randomly sampling θᵢ for each i of Cᵢ ∈C^{P}, from a normal distribution N(\hat{θ}ᵢ, αₜ²Bᵢ⁻¹) wherein \hat{θ}ᵢ is a vector of order N and Bᵢ is an N×N matrix, and wherein said updating comprises updating \hat{θ}ᵢ and Bᵢ (i=1, . . . , N) in each iteration t<T; and
selecting C^{U}(t) from Cᵢ^{U} to maximize Σ_{i∈{i}} c^{V}(t)^{T}θᵢ, wherein Cᵢ^{U}=U features Cᵢ in C^{P} and {i}=indexes i of the U features Cᵢ in C^{P}.

20. The computer system of claim 16, wherein the method comprises in iteration t:
randomly sample μₖ for k=1, . . . , K from a normal distribution N(\hat{μ}ₖ, αₜ²Aₖ⁻¹), wherein μₖ is a vector of order N and Aₖ is an N×N matrix, and wherein said updating comprises updating μₖ and Aₖ in each iteration t<T; and
selecting the arm k(t) from the K arms to maximize c^{U+V}(t)^{T}μₖ.
